(19) United States
(12) Patent Application Publication
(10) Pub. No.: US 2026/0345959 A1
(43) Pub. Date: Nov. 5, 2026

(54) Polymeric Coatings

(71) Applicant: MicroVention, Inc., Aliso Viejo, CA (US)

(72) Inventors: Belete Degaga, Mission Viejo, CA (US); Joseph Gulachenski, Trabuco Canyon, CA (US); Nicholas Davis, Mission Viejo, CA (US); Barry Tan, Cypress, CA (US); Tadele Wolde-Meskel, Anaheim, CA (US)

(73) Assignee: MicroVention, Inc., Aliso Viejo, CA (US)

(21) Appl. No.: 19/487,868

(22) PCT Filed: May 30, 2024
(86) PCT No.: PCT/US2024/031772

§ 371 Date: Nov. 25, 2025

Related U.S. Application Data
(60) Provisional application No. 63/505,364, filed on May 31, 2023.

(51) Int. Cl. A61M 25/09

(52) U.S. Cl. CPC A61M 25/09; A61M 2025/09083; A61M 2025/09108; A61M 2025/09141; A61M 2205/0238; A61M 2207/10

(57) ABSTRACT

A polymeric coating for medical devices. The polymeric coating may be formed by dissolving polymer solids in a liquid solvent and mixing the solids and liquid solvent together to form a homogeneous polymeric solution. The polymeric solution may be applied to at least a portion of a medical device and allowed to cure or dry to form a polymeric jacket or coating on the medical device. Alternatively, the polymeric solution may be cured or dried separately and then applied to the medical device. Various polymers may be utilized, including but not limited to polyurethane and/or polycarbonate based biopolymers. Various solvents may be utilized, including but not limited to dimethylacetamide.

### RELATED APPLICATIONS

This application claims benefit of and priority to U.S. Patent Application Ser. No.: 63/505,364 filed May 31, 2023 and entitled Polymeric Coatings, which is hereby incorporated herein by reference in its entirety.

### BACKGROUND OF THE INVENTION

Many modern medical devices may include multiple components which are secured together to form a unitary body. As a non-limiting example, certain guidewires may include one or more coils wound around a central core wire. To facilitate torquability, trackability, and shapeability of such medical devices, it may be desirable to reinforce and connect such coils to the core wire to form a continuous, smooth surface.

One method of forming such a continuous, smooth surface may be the use of various jackets which coat and encapsulate the various components of various types of medical devices. The use of such jackets may be desirable to connect such various components in a manner that maintains a uniform torque response when a rotational force is applied (e.g., one-to-one torquability or proximal-to-distal rotational ratio).

It may also be desirable to utilize a substance to form such a jacket which keeps such encapsulated components intact and flexible, even while tracking through a tortuous anatomical path without unwinding or otherwise becoming structurally compromised. As a non-limiting example, it may be desirable to utilize a jacket which encapsulates one or more coils with an underlying core wire such that the coils do not unwind or lose adhesion during while traversing a tortuous pathway.

### SUMMARY OF THE INVENTION

In some aspects, a polymeric coating may be applied to a medical device in a liquid form and then allowed to dry or cure to form a solid, continuous surface.

In some aspects, the polymeric coating may be composed of a polyurethane based substance.

In some aspects, the polymeric coating may be composed of a polycarbonate based substance.

In some aspects, a polymeric coating may be formed from a solvent and a polymer (e.g., polyurethane based and/or polycarbonate based) dissolved in the solvent.

In some aspects, a polyurethane based polymer may be comprised of an aliphatic hydrocarbon thermoplastic polyurethane based solution.

In some aspects, a solvent may be composed of dimethylacetamide (DMAC).

In some aspects, a volume of the polymer may be between about 15%-25% of a total volume of a resulting polymeric coating.

In some aspects, a volume of the solvent may be between about 75%-85% of a total volume of a resulting polymeric coating.

In some aspects, a solvent may be comprised of tetrahydrofuran, methoxy propyl acetate, and/or toluene.

In some aspects, a medical device may comprise a polymeric coating which coats the medical device to form a smooth, continuous surface.

In some aspects, such a medical device may comprise a guide wire formed from a core wire and one or more coils wound around the core wire; with the polymeric coating encapsulating the core wire and the one or more coils.

In some aspects, such a medical device may comprise a guide wire formed from a core wire, an inner coil, and an outer coil; with the polymeric coating encapsulating the core wire, the inner coil, and the outer coil to form a smooth, continuous surface.

In some aspects, the core wire may be composed of Nitinol, the first coil may be composed of stainless steel, and the second coil may be composed of platinum tungsten.

In some aspects, such a medical device may be comprised of a stent which is at least partially coated with a polymeric coating.

In some aspects, a method of fabricating a polymer coated medical device may comprise diluting a polymer in a solvent to form a solution, mixing the solution, applying the solution to coat at least a portion of a medical device, and curing or drying the solution.

In some aspects, the step of applying the solution to coat at least a portion of a medical device may comprise use of a container including an upper opening and a lower opening, with the upper opening being wider than the lower opening.

In some aspects, the step of applying the solution to coat at least a portion of a medical device may comprise inserting the medical device at least partially through a container such that the medical device extends through both an upper opening and a lower opening of the container.

In some aspects, the step of applying the solution to coat at least a portion of a medical device may comprise at least partially filling the container with a solution comprised of a polymer diluted in a solvent.

In some aspects, the step of applying the solution to coat at least a portion of a medical device may comprise removing the medical device from the container through the upper opening such that the solution coats at least a portion of the medical device.

In some aspects, a slitted polymer foam or sponge may be attached to the lower opening of the container to prevent leaks.

In some aspects, the solution may be dried by hanging the coated medical device on a rack.

In some aspects, the solution may be dried by applying heat, such as by circulating warm arm in a heated oven, to the medical device after the solution has been applied thereto.

In some aspects, the techniques described herein relate to a polymeric coating for a medical device, including: a solvent; and a polyurethane-based polymer dissolved in the solvent.

In some aspects, the techniques described herein relate to a polymeric coating, wherein the polyurethane-based polymer includes an aliphatic hydrocarbon thermoplastic polyurethane-based solution.

In some aspects, the techniques described herein relate to a polymeric coating, wherein the solvent includes dimethylacetamide.

In some aspects, the techniques described herein relate to a polymeric coating, wherein a volume of the polyurethane-based polymer is between about 15%-25% of a total volume of the polymeric coating.

In some aspects, the techniques described herein relate to a polymeric coating, wherein a volume of the solvent is between about 75%-85% of a total volume of the polymeric coating.

In some aspects, the techniques described herein relate to a polymeric coating, wherein a volume of the polyurethane-based polymer is about 20% of a total volume of the polymeric coating.

In some aspects, the techniques described herein relate to a polymeric coating, wherein a volume of the solvent is about 80% of the total volume of the polymeric coating.

In some aspects, the techniques described herein relate to a polymeric coating, wherein the solvent includes tetrahydrofuran.

In some aspects, the techniques described herein relate to a polymeric coating, wherein the solvent includes methoxy propyl acetate.

In some aspects, the techniques described herein relate to a polymeric coating, wherein the solvent includes toluene.

In some aspects, the techniques described herein relate to a polymeric coating for a medical device, including: a solvent included of dimethylacetamide; and a polymer included of an aliphatic hydrocarbon thermoplastic polyurethane; wherein a volume of the polymer is between 15%-25% of a total volume of the polymeric coating; and, wherein a volume of the solvent is between 75%-85% of the total volume of the polymeric coating.

In some aspects, the techniques described herein relate to a coated medical device system, including: a medical device; and a polyurethane-based polymeric solution applied to at least a portion of the medical device.

In some aspects, the techniques described herein relate to a coated medical device system, wherein the medical device includes a guidewire.

In some aspects, the techniques described herein relate to a coated medical device system, further including a first coil coiled around at least a portion of a length of the guidewire.

In some aspects, the techniques described herein relate to a coated medical device system, further including a second coil coiled around at least a portion of the length of the guidewire.

In some aspects, the techniques described herein relate to a coated medical device system, wherein the first coil includes an inner coil and wherein the second coil includes an outer coil.

In some aspects, the techniques described herein relate to a coated medical device system, wherein the guidewire is composed of Nitinol.

In some aspects, the techniques described herein relate to a coated medical device system, wherein the first coil is composed of stainless steel.

In some aspects, the techniques described herein relate to a coated medical device system, wherein the second coil is composed of platinum tungsten.

In some aspects, the techniques described herein relate to a coated medical device system, wherein the medical device includes a stent.

In some aspects, the techniques described herein relate to a method of fabricating a polymer coated medical device, including: diluting a polyurethane-based polymer in a solvent to form a solution; mixing the solution; applying the solution to at least a portion of a medical device; and drying the solution.

In some aspects, the techniques described herein relate to a method, wherein the step of applying the solution includes: providing a container including an upper opening and a lower opening, the upper opening being wider than the lower opening; inserting the medical device at least partially through the container such that the medical device extends through both the upper opening and the lower opening; at least partially filling the container with the solution; and removing the medical device from the container through the upper opening such that the solution coats at least a portion of the medical device.

In some aspects, the techniques described herein relate to a method, further including the step of attaching a slitted polymer foam or sponge to the lower opening of the container for leak prevention.

In some aspects, the techniques described herein relate to a method, wherein the step of drying the solution includes hanging the medical device in a rack.

In some aspects, the techniques described herein relate to a method, wherein the step of drying the solution includes applying heat to the medical device after the solution has been applied.

In some aspects, the techniques described herein relate to a method, wherein the polyurethane-based polymer includes an aliphatic hydrocarbon thermoplastic polyurethane-based solution.

In some aspects, the techniques described herein relate to a method, wherein the solvent includes dimethylacetamide.

In some aspects, the techniques described herein relate to a method, wherein a volume of the polyurethane-based polymer is between 15%-25% of a total volume of the solution.

In some aspects, the techniques described herein relate to a method, wherein a volume of the solvent is between 75%-85% of a total volume of the solution.

In some aspects, the techniques described herein relate to a method, wherein a volume of the polyurethane-based polymer is 20% of a total volume of the solution.

In some aspects, the techniques described herein relate to a method, wherein a volume of the solvent is 80% of the total volume of the solution.

In some aspects, the techniques described herein relate to a method, wherein the solvent includes tetrahydrofuran.

In some aspects, the techniques described herein relate to a method, wherein the solvent includes methoxy propyl acetate.

In some aspects, the techniques described herein relate to a method, wherein the solvent includes toluene.

In some aspects, the techniques described herein relate to a method, wherein the medical device includes a guidewire.

In some aspects, the techniques described herein relate to a method, further including a first coil coiled around at least a portion of a length of the guidewire.

In some aspects, the techniques described herein relate to a method, further including a second coil coiled around at least a portion of the length of the guidewire.

In some aspects, the techniques described herein relate to a method, wherein the first coil includes an inner coil and wherein the second coil includes an outer coil.

In some aspects, the techniques described herein relate to a method, wherein the guidewire is composed of Nitinol.

In some aspects, the techniques described herein relate to a method, wherein the first coil is composed of stainless steel.

In some aspects, the techniques described herein relate to a method, wherein the second coil is composed of platinum tungsten.

In some aspects, the techniques described herein relate to a method, wherein the medical device includes a stent.

In some aspects, the techniques described herein relate to a polymeric coating for a medical device, including: a solvent; and a polycarbonate-based polymer dissolved in the solvent.

In some aspects, the techniques described herein relate to a medical device, wherein the first coil includes an inner coil and wherein the second coil includes an outer coil.

In some aspects, the techniques described herein relate to a medical device, wherein the first coil includes a variable pitch along its length.

In some aspects, the techniques described herein relate to a medical device, wherein the first coil includes a first segment having a first pitch, a second segment having a second pitch, and a third segment having a third pitch.

In some aspects, the techniques described herein relate to a medical device, wherein the first pitch is 0.0005 inch, the second pitch is 0.008 inch, and the third pitch is 0.013 inch.

In some aspects, the techniques described herein relate to a medical device, wherein the first coil includes a fourth segment having a fourth pitch and wherein the fourth pitch is 0.00175 inch.

In some aspects, the techniques described herein relate to a medical device, wherein the fourth segment is proximal with respect to the first segment, the second segment, and the third segment.

In some aspects, the techniques described herein relate to a medical device, wherein the second coil includes a uniform pitch along its length.

In some aspects, the techniques described herein relate to a medical device, wherein the uniform pitch of the second coil is 0.00175 inch.

In some aspects, the techniques described herein relate to a medical device, further including a polymeric coating encapsulating the first coil and the second coil.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1A is a partial side view of a medical device coated in a polymeric coating according to one example.

FIG. 1B is a partial side sectional view of a medical device coated in a polymeric coating according to one example.

FIG. 1C is a partial side sectional view of a medical device including inner and outer coils coated in a polymeric coating according to one example.

FIG. 1D is a partial side view of a medical device including an inner coil and an outer coil according to one example.

FIG. 1E a partial side view of a medical device including an inner coil and an outer coil according to one example.

FIG. 1F a partial side view of a medical device including an inner coil and an outer coil according to one example.

FIG. 2 is a perspective view of a container for coating a medical device with a polymeric coating according to one example.

FIG. 3 is a perspective view of a container including a leak guard for coating a medical device with a polymeric coating according to one example.

FIG. 4 is a perspective view of a medical device inserted through a container prior to being coated with a polymeric coating according to one example.

FIG. 5 is a perspective view of a polymer solution introduced into a container so as to coat a medical device with a polymeric coating according to one example.

FIG. 6 is a perspective view illustrating removal of the medical device from the container to coat the medical device with a polymeric coating according to one example.

FIG. 7 is a flowchart illustrating a method of coating a medical device with a polymeric coating according to one example.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

Specific aspects of the invention will now be described with reference to the accompanying drawings. This invention may, however, be embodied in many different forms and should not be construed as limited to the examples set forth herein; rather, these examples are provided so that this disclosure will be thorough and complete, and will fully convey the scope of the invention to those skilled in the art. The terminology used in the detailed description of the examples illustrated in the accompanying drawings is not intended to be limiting of the invention. In the drawings, like numbers refer to like elements.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 10% of a stated value, inclusive.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Disclosed herein are various examples of a polymeric coating which may be utilized to coat at least a portion of a wide range of medical devices. Polymeric coatings may provide superior adherence and protection from corrosion.

Polymeric coatings may be utilized to coat, treat, be applied to, and/or encapsulate various components of such medical devices for a wide range of purposes. The polymeric coating process may comprise application of an elastomer or other polymeric material onto a support substrate.

The use of a polymeric coating on a medical device may provide a number of advantages or benefits. In medical devices having coils such as described below, the polymeric coating may reinforce and connect the coils to form a continuous surface so as to make the coils intact and flexible while tracking through a tortuous anatomical path. The polymeric coating may also facilitate continuous torque transmission and prevent coil misalignment. The polymeric coating may be utilized to form an extruded polymer jacket which may be applied to a medical device.

By way of example and without limitation, the various examples of the polymeric coating shown and/or described herein may be utilized in connection with medical devices including delivery devices, occlusive devices, tubular devices, catheters, guidewires, stents, shunts, hypotubes, and the like.

Polymeric coatings may comprise a solvent and a polymer dissolved in the solvent. Dissolving the polymer in the solvent may aid in reducing the viscosity of the polymer so that it forms a liquid that may be applied to a medical device. The rheology of the resulting coating solution may be tailored to the coating and drying method by varying the amount of solvent in the solution. Once the liquid solution is applied to a substrate, the solvent may be removed in a drying or curing process.

As the solvent is removed, the glass transition temperature of the solution may increase. Nominally, when the glass transition temperature is greater than the drying temperature, the coating may be solid. If the drying temperature is higher than room temperature, the coating may continue to solidify or harden upon cooling. Additionally, some polymers may crystallize as solvent is removed or on cooling from the drying temperature, thereby creating a final polymer coating that is semicrystalline.

Various factors may have an effect on adherence of the polymeric coating to an underlying substrate, such as a portion or component of a medical device. Contact angle and wetting behavior of solid particles may be influenced by many physical and chemical factors such as surface roughness and heterogeneity as well as particle shape and size. Surface geometry, and surface roughness, can be used to tune the contact angle, with increasing surface roughness decreasing the apparent contact angle for hydrophilic materials and adhering the coating to the substrate.

The polymer may comprise a wide range of polymers, including but not limited to biopolymers such as polyurethane-based polymers and polycarbonate-based polymers. By way of example, a polymeric coating may comprise an aliphatic hydrocarbon thermoplastic polyurethane-based solution, such as a polymer jacket, that may be extruded to various forms, including a tubular form. In other examples, polymers such as various thermoplastic polyether polyurethanes, thermoplastic polycarbonate polyurethanes, thermoplastic silicone polycarbonate polyurethanes, segments polyester polyurethanes, thermoplastic polyether polyurethanes, and/or thermoplastic silicone polyether polyurethanes may be utilized.

Polyurethane-based polymers may comprise aliphatic hydrocarbons (e.g., a hydrocarbon compound comprising carbon and hydrogen joined together in straight chains, branched trains, or non-aromatic rings) so as to include urethane linkages. Polyurethane may provide improved flexibility, low density, durability, and the like.

Polycarbonate-based polymers may comprise aromatic hydrocarbons (e.g., unsaturated hydrocarbons which have one or more planar six-carbon rings, commonly referred to as benzene rings or aromatic rings, to which hydrogen atoms may be attached) so as to include carbonate groups. Polycarbonate may provide high impact resistance, low scratch resistance, transparency, resistance to elevated temperatures, and the like.

Various types of solvents may be utilized for forming the solution with the polymer. By way of example, suitable solvents may include dimethylacetamide (“DMAC”), tetrahydrofuran, methoxy propyl acetate, toluene, dimethylformamide, dimethyl sulfoxide, dichloromethane, chloroform, hexafluoro isopropanol, tetrahydrofuran, combinations thereof, and the like.

As previously mentioned, the volume ratio of the polymer and the solvent may vary in different examples to provide different characteristics. By way of example, the volume of the polymer may be between about 15%-25% of a total volume of the polymeric coating and the volume of the solvent may be between about 75%-85% of the total volume of the polymeric coating. As a further example, the volume of the polymer may be about 20% of a total volume of the polymeric coating and the volume of the solvent may be about 80% of the total volume of the polymeric coating.

Disclosed herein are various examples of a coated medical device system. The coated medical device system may comprise a medical device and a polymeric solution applied to at least a portion of the medical device. As previously mentioned, the polymeric solution may be applied to a wide range of medical devices which would benefit from a polymeric coating. The polymeric solution may be applied to all or a portion of such medical devices.

The medical device may comprise a guidewire. The guidewire may comprise a core wire and at least one coil wound around at least a portion of a length of the guidewire. The at least one coil may be comprised of a first coil coiled around at least a portion of the length of the guidewire and a second coil coiled around at least a portion of the length of the guidewire. The first coil may comprise an inner coil and the second coil may comprise an outer coil which is coiled over and around the inner coil. The core wire may be composed of various materials, such as Nitinol. The coil(s) may be composed of various materials, such as stainless steel, platinum tungsten, and the like.

Disclosed herein are various examples of a method of forming a polymeric solution that may be applied to a medical device. By way of example, a method of forming a polymeric solution may comprise providing a polymer, providing a solvent, and dissolving the polymer in the solvent.

Disclosed herein are various examples of a method of fabricating a polymer coated medical device. By way of example, a method of fabricating a polymer coated medical device may comprise diluting a polymer in a solvent to form a solution, mixing the solution, applying the solution to at least a portion of the medical device, and drying the solution.

Disclosed herein are various examples of a method of applying a polymeric solution to a medical device. By way of example, a method of applying a polymeric solution to a medical device may comprise providing a container including an upper opening and a lower opening, inserting the medical device at least partially through the container such that the medical device extends through both the upper and lower openings of the container, at least partially filling the container with a polymeric solution, and removing the medical device from the container through the upper opening such that the solution coats at least a portion of the medical device. A leak preventer, such as a slitted polymer foam or sponge, may be attached to the lower opening of the container for leak prevention. Drying the solution may be comprised of hanging the coated medical device in a rack, applying heat, applying light, and the like.

Specific examples are described further below. However, it should be understood that any of the features from any of the examples can be mixed and matched with each other in any combination. Hence, the present disclosure should not be restricted to only these examples, but any broader combination thereof.

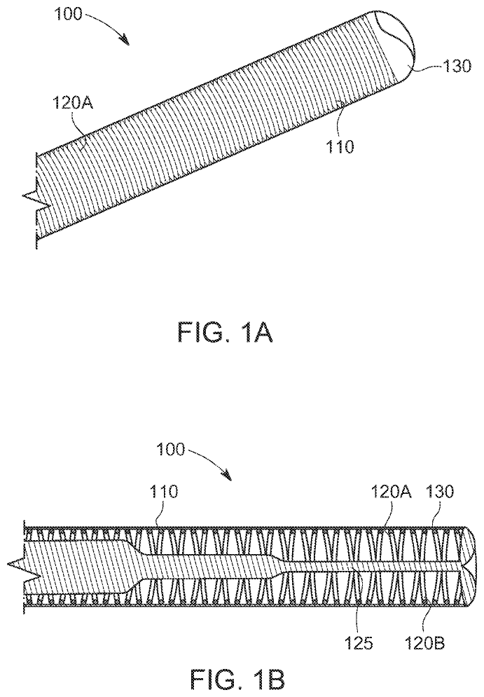

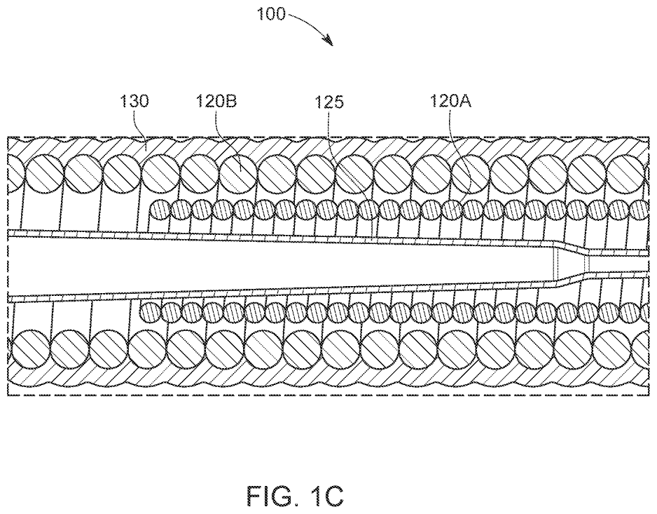

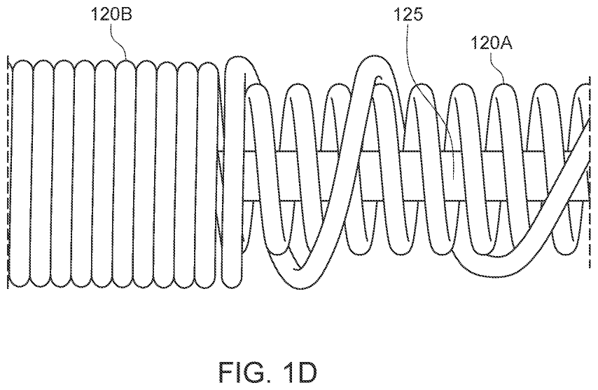

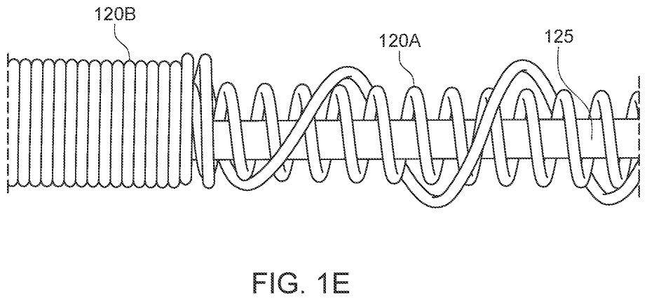

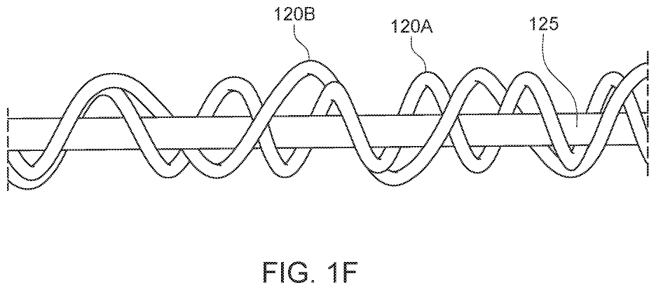

FIG. 1A is a partial side view of a medical device 110 coated in a polymeric coating 130 according to one example. In the example shown in FIG. 1A, the medical device 110 is illustrated as comprising a delivery device having a polymeric coating 130 which surrounds and encapsulates at least one coil 120A. It should be appreciated that a wide range of medical devices, including delivery devices, such as but not limited to a guidewire, pusher, or catheter, may benefit from the systems, devices, and/or methods shown and/or described herein. The medical device 110 may comprise a blunted or rounded distal tip as shown.

While FIG. 1A illustrates an example medical device, it should be appreciated that the medical device 110 may comprise a wide range of medical devices, including medical devices which would benefit from application of a polymeric coating for any number of purposes. The scope of the systems and/or methods which are shown and/or described herein should not be construed as being limited to any particular type of medical device 110. Merely for exemplary purposes, the medical device 110 may comprise a delivery device, an occlusive device, a tubular device, a guiding device, various catheters, guidewires, shunts, stents, hypotubes, and the like.

FIG. 1B is a partial side sectional view of a medical device 110 coated in a polymeric coating 130 according to one example. The medical device 110 may comprise a guidewire which includes a core wire 125 and one or more coils 120A, 120B. The one or more coils 120A, 120B may be connected to at least a portion of a length of the core wire 125. In some examples, the one or more coils 120A, 120B may be wound or coiled around at least a portion of the length of the core wire 125. While the figures illustrate a pair of coils 120A, 120B, it should be appreciated that more (e.g., three or more) or less (e.g., one) coil(s) 120A, 120B may be utilized. Additionally, as discussed in more detail below, the winding configuration, orientation, pitch, positioning, and/or spacing of the one or more coils 120A, 120B may vary in different examples and thus should not be construed as limited by the examples shown in the figures.

In some examples, the medical device 110 may comprise a guidewire with grooves, notches, or other types of indentations which perform a similar function to such coils. In such examples, separate coil(s) may be omitted. Examples of such guidewires are shown and described in U.S. patent application Ser. No. 63/386,422, filed on Dec. 7, 2022 and PCT Application No. PCT/US2023/082954, filed on Dec. 7, 2023, both of which are hereby incorporated by reference in their entireties.

FIG. 1C is a partial side sectional view of a medical device 110 including inner and outer coils 120A, 120B coated in a polymeric coating according to one example. The medical device 110 may comprise a core wire 125 around which is wound, coiled, or otherwise connected at least an inner coil 120A and an outer coil 120B. A polymeric coating 130 may be applied to coat and encapsulate the core wire 125 and both coils 120A, 120B to provide a smooth, continuous surface for at least a portion of the length of the medical device 110. However, it should be appreciated that the polymeric coating 130 may be applied to a medical device 110 having only a single coil, or a medical device 110 having more than two coils.

Continuing to reference FIG. 1C, it can be seen that, in an example, the inner coil 120A may be at least partially positioned within the inner circumference of the outer coil 120B. The respective lengths of the inner coil 120A and the outer coil 120B may vary in different examples and thus should not be construed as limited by the examples shown in the figures. In some examples, the inner coil 120A may be longer than the outer coil 120B. In other examples, the outer coil 120B may be longer than the inner coil 120A. In yet other examples, both the inner and outer coils 120A, 120B may be substantially the same length. In an example, the length of the outer coil 120B may be between about 30 cm and 40 cm.

The inner coil 120A may be composed of the same material as the outer coil 120B or the inner coil 120A may be composed of a different material than the outer coil 120B. In an example, the inner coil 120A may be composed of stainless steel and the outer coil 120B may be composed of platinum tungsten. The outer coil 120B may thus be radiopaque along its entire length (e.g., the outer coil 120B may provide a continuous radiopacity along its entire length). In some examples, however, only a portion of the length of the outer coil 120B may be radiopaque.

The respective filar dimensions of the inner and outer coils 120A, 120B may similarly be substantially the same or different with respect to each other. In an example, the filar outer diameter of the inner and outer coils 120A, 120B may each be about 0.00125 inch.

The winding directions of the inner and outer coils 120A, 120B may be the same in some examples, or may be different from each other. In an example, the inner coil 120A may be wound in a first direction and the outer coil 120B may be wound in a second direction, with the first direction being opposite to the second direction. In an example, the inner coil 120A may be wound in a clockwise direction and the outer coil 120B may be wound in a counterclockwise direction. In another example, the inner coil 120A may be wound in a counterclockwise direction and the outer coil 120B may be wound in a clockwise direction. In yet another example, both the inner and outer coils 120A, 120B may be wound in a clockwise direction or a counterclockwise direction.

FIG. 1D is a partial side view of a medical device 110 including an inner coil 120A and an outer coil 120B according to one example. As shown, the inner coil 120A may comprise an outer diameter which is less than an outer diameter of the outer coil 120B such that the inner coil 120A lies within the outer coil 120B. The inner coil 120A may comprise a uniform winding pattern or pitch along its length as shown or may comprise a variable winding pattern or pitch in some examples. The outer coil 120B may comprise a variable winding pattern or pitch as shown or may comprise a uniform winding pattern or pitch in some examples. In the example shown in FIG. 1D, it can be seen that the outer coil 120B may be more tightly wound along a portion of its length, and more loosely wound around another portion of its length.

In an example, one or more portions of the inner coil 120A may comprise a pitch between about 0.00175 inch and 0.013 inch. In an example, a proximal portion of the inner coil 120A may comprise a pitch of about 0.00175 inch. In an example, a distal portion of the inner coil 120A may comprise a pitch of between about 0.005 inch and 0.013 inch.

The pitch of the inner coil 120A may vary along its length. In an example, a pitch of the inner coil 120A may vary along a proximal portion of its length; with a first segment of the proximal portion comprising a pitch of about 0.005 inch, a second segment of the proximal portion comprising a pitch of about 0.008 inch, and a third segment of the proximal portion comprising a pitch of about 0.013 inch. The pitch of a distal portion of a length of the inner coil 120A may similarly vary in some examples.

The pitch of the outer coil 120B may be uniform along its length or may vary along its length. In an example, an entire length of the outer coil 120B may comprise a pitch of about 0.00175 inch.

FIG. 1E is a partial side view of a medical device 110 including an inner coil 120A and an outer coil 120B according to one example. FIG. 1E illustrates an example in which the outer coil 120B may comprise a tighter winding pattern or pitch along a portion of its length and a looser, helical winding pattern or pitch along another portion of its length, with a uniform inner coil 120A lying within the windings of the outer coil 120B and the core wire 125 lying within the windings of the inner coil 120A.

FIG. 1F is a partial side view of a medical device 110 including an inner coil 120A and an outer coil 120B according to one example. FIG. 1F illustrates an example in which both the inner and outer coils 120A, 120B comprise a looser winding or pitch than the previous examples shown in FIGS. 1D and 1E. As shown, the inner coil 120A may comprise a tighter winding pattern or pitch than the outer coil 120B. However, in some examples, the reverse configuration may be utilized (e.g., the inner coil 120A may comprise a looser winding pattern or pitch than the outer coil 120B) or both the inner and outer coils 120A, 120B may comprise substantially the same winding pattern or pitch dimensions.

In some examples, the coils 120A, 120B may have variable winding rates such that, depending on the portion of the length of the core wire 125, the coils 120A, 120B may alternate between being “inner” or “outer” with respect to each other. Thus, the first coil 120A may weave in and out of the second coil 120B such that, depending on the portion of the core wire 125 being referenced, the first coil 120A may comprise an “inner” or an “outer” coil with respect to the second coil 120B.

While FIGS. 1D-1F omit the polymeric coating 130 so as to better visualize the coils 120A, 120B, it should be appreciated that the polymeric coatings 130 shown and/or described herein may be applied to the coils 120A, 120B of the examples shown in FIGS. 1D-1F using the methods shown and/or described herein so as to provide a continuous smooth surface for the medical device 110. The use of such a polymeric coating 130 may be particularly beneficial in such medical devices 110 in which multiple coils 120A, 120B comprise different winding patterns or pitches so as to facilitate torquability, trackability, and shapeability and/or to reinforce and connect such coils 120A, 120B to the core wire 125 to form a continuous, smooth surface.

The polymeric coating 130 may aid in facilitating torquability, trackability, and shapeability of such medical devices 110 by reinforming and connecting the coils 120A, 120B to create a continuous smooth surface. The polymeric coating 130 may form a reinforcing polymer jacket that strongly connects the coils 120A, 120B both together and to the underlying core wire 125 such that the torque may be transmitted from proximal end to distal tip and thereby maintain a uniform torque response when a rotational force is applied on the proximal end of the medical device 110 (e.g., one-to-one torquability). The polymeric coating 130 may also function to hold and retain the respective positions and orientations of the core wire 125, inner coil 120A, and/or outer coil 120B, even when traversing through tortuous anatomies.

In an example, the polymeric coating 130 may comprise a solution formed from at least a solvent and a polymer. In an example, the solution may be comprised of a polyurethane-based polymer, such as an aliphatic hydrocarbon thermoplastic polyurethane-based solution. In another example, the solution may be comprised of a polycarbonate-based polymer. In yet other examples, the solution may be comprised of both a polycarbonate-based polymer and a polyurethane-based polymer.

The polymeric coating 130 may be formed from polymers that provide superior adherence and protection from corrosion. Polymeric processes such as those shown and/or described herein may apply an elastomer or other polymeric material onto a supporting substrate, such as a medical device 110.

To reduce the viscosity of a polymer so as to become a coatable liquid, the polymer may be dissolved in the solvent and thereby form a liquid that may be applied to various substrates. The rheology of the coating solution may be tailored to various coating and/or drying methods by altering the amount of solvent in the solution.

The manner by which the polymer is dissolved in the solvent to form the solution may vary in different examples. In one example, the polymer may be introduced into the solvent and then both the polymer and the solvent may be agitated in various manners to dissolve the solvent. For example, the polymer may be dissolved in the solvent by mixing or shaking the polymer within the solvent until the solution is formed.

Various types of solvents known to dissolve polymers may be utilized. By way of example, various polar organic solvents may be utilized. In an example, the solvent may be comprised of dimethylacetamide (“DMAC”). However, various other solvents may be utilized, alone or in combination, such as but not limited to tetrahydrofuran, methoxy propyl acetate, toluene, dimethylformamide, dimethyl sulfoxide, dichloromethane, chloroform, hexafluoro isopropanol, tetrahydrofuran, combinations thereof, and the like. In another example, the solvent may be comprised of a combination of any or all of the aforementioned substances.

The ratio of the volumes of the polymer and the solvent used to form the polymeric solution may vary in different examples. Put differently, the amount of polymer or solvent utilized, as a percentage of the total volume of the resulting solution, may vary to suit different applications or examples. For example, the respective amounts of polymer and solvent used may affect the consistency, viscosity, and/or elasticity of the resulting solution.

The polymeric solution formed by diluting the polymer and the solvent may have a “mix-up ratio” defined by the percentage of the volume of the polymer as compared to the total volume of the resulting solution (i.e., the sum of the volumes of the polymer and the solvent). In an example, the mix-up ratio, or the percentage of the polymer as compared to the total volume of the polymeric solution, may comprise between about 15%-25%. Thus, the total dissolved solids (TDS) value of the solution, referring to the concentration of the polymer in the solution, may be between about 15%-25%. However, in some examples, TDS values of less than 15% may be utilized such as, for example, a liquid solution including a polymer soluble in tetrahydrofuran.

Thus, a volume of the polymer may be between about 15%-25% of a total volume of the polymeric coating or solution. A volume of the solvent may be between about 75%-85% of the total volume of the polymeric coating or solution. However, as previously mentioned, these values may vary. In some examples, the volume of the solvent may comprise 50% or more of a total volume of the polymeric coating or solution. In other examples, the volume of the polymer may comprise 50% or more of a total volume of the polymeric coating or solution. In yet other examples, the volume of the solvent may comprise 50% or less of a total volume of the polymeric coating or solution. In yet other examples, the volume of the polymer may comprise 50% or less of a total volume of the polymeric coating or solution.

It should be appreciated that the resulting solution may be utilized in a number of different manners to suit different applications. In an example, the solution may be applied to a medical device as a liquid so as to coat, treat, or otherwise cover all or at least a portion of the medical device. In another example, the solution may be extruded into various shapes, such as a tubular shape, and then cured or dried to form a polymeric jacket or coating that may be attached in various manners to different types of medical devices. Thus, the solution may be solidified directly on the medical device, or it may be formed into a desired shape, allowed to solidify, and then applied to the medical device.

The manner or method of formation of the polymeric solution or coating may vary in different examples. In an example, the polymer may first be diluted in the solvent, such as by introducing a volume of the polymer into a volume of the solvent. Exemplary methods of introducing the polymer into the solvent include, but are not limited to, injection, pouring, spraying, or the like.

The polymer may initially be in a solid state. By way of example, the polymer may comprise pellets prior to being dissolved in the solvent. The solid polymer may be dried prior to dissolving in a solvent. In an example, the polymer may be dried to have less than about 0.05% by weight moisture content. In an example, the polymer, in a solid state, may be dried in a desiccant type de-humidifying dryer capable of reaching and maintaining a dew point of between about −4 to-58 degrees Fahrenheit at a temperature of between about 140 to 158 degrees Fahrenheit for about 3-5 hours.

Generally, the liquid solution may be formed by mixing the polymer and the solvent together. The manner or method of mixing the polymer and solvent may vary in different examples. In an example, the polymer and solvent may be agitated in various manners to mix the polymer and solvent together to form an integrated liquid solution. Exemplary methods of mixing include, but are not limited to, stirring, shaking, use of a stirrer plate and/or stirrer bar, and various other methods known for mixing two components of a solution together.

In an example, the polymer in a solid state may be dissolved by being added to the solvent in a container such as a glass jar while continuously being agitated to remove bubbles, such as by continuous stirring with a mechanical mixer, a planetary centrifugal mixer, and the like. A stirrer such as a dissolving or dispersion type stirrer may be utilized to prevent the polymer solids from clumping at the bottom of the container, which can prolong dissolution time. Although stirring speed may vary, high RPM's (e.g., between about 750-1000 RMP) may be desirable to improve efficiency during dissolution.

The amount of time necessary for the dissolution process to be completed, resulting in a homogeneous liquid solution, may vary. In an example having about a 20% solids content, the dissolution process may be completed after about 3-4 hours of mixing or agitation.

After agitation, but prior to application, the solution may be filtered. By way of example, the solution may be filtered through stainless steel or polypropylene pressured filters having mesh sizes that are less than or equal to about 20 microns. The solution may be filtered with nitrogen pressure, with the requisite amount of pressure depending on the pressure rating of the type of filter used, as well as the solvent concentration.

After the solution has been formed, it may either be applied directly to a medical device or extruded into a desired shape and then applied, such as by attachment, to a medical device. In some examples, the extruded solution may itself form all or part of the medical device.

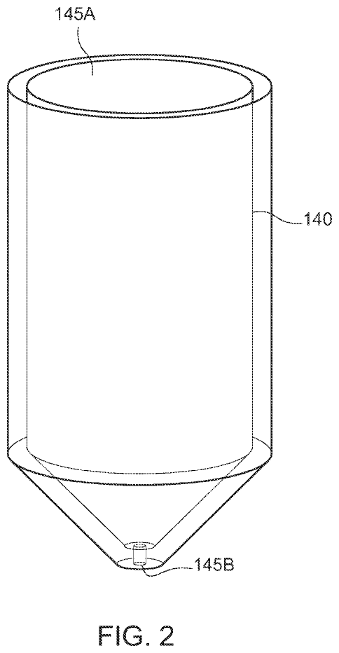

FIG. 2 is a perspective view of a container for coating a medical device with a polymeric coating according to one example. As shown in FIG. 2, a container 140 such as a crucible may be utilized for applying the solution 135 to the medical device 110. The container 140 may comprise an upper opening 145A and a lower opening 145B. However, in some examples, the openings may instead be arranged on the sides of the container 140, in which case the container 140 may comprise a first side opening and a second side opening. The container 140 may comprise a partially tapered body, with a lower portion of the container 140 being tapered such that the upper opening 145A is wider than the lower opening 145B.

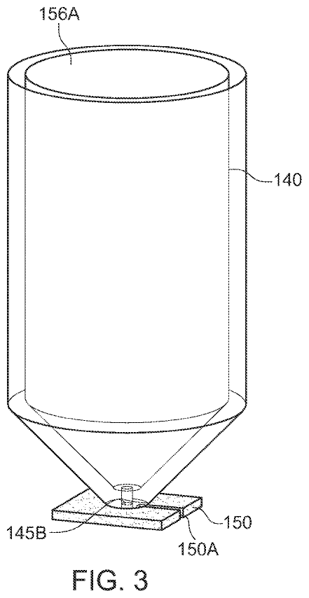

FIG. 3 is a perspective view of a container including a leak guard for coating a medical device with a polymeric coating according to one example. As shown in FIG. 3, a leak preventer 150 may be attached to the bottom of the container 140, such as to or near the lower opening 145B thereof. The leak preventer 150 may comprise, e.g., a polymer foam or sponge such as shown in FIG. 3. The leak preventer 150 may include a slit 150A to guide any leaking solution.

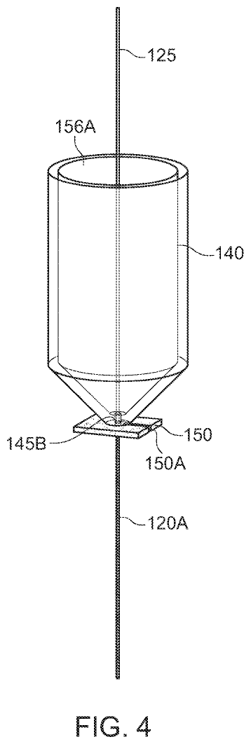

FIG. 4 is a perspective view of a medical device inserted through a container prior to being coated with a polymeric coating according to one example. As shown in FIG. 4, the medical device 110 may be inserted into the cavity of the container 140. In the example shown in FIG. 4, in which the medical device 110 may be comprised of a guidewire having one or more coils 120A, 120B along a portion of its length, the guidewire may be inserted into the container 140 until the coiled section of its length protrudes through and past the lower opening 145B such as shown in FIG. 4.

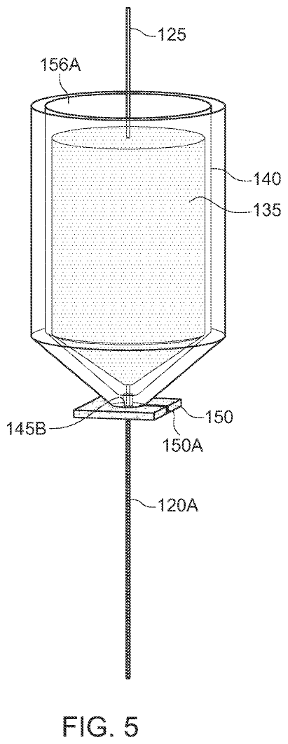

FIG. 5 is a perspective view of a polymer solution introduced into a container so as to coat a medical device with a polymeric coating according to one example. As shown in FIG. 5, a diluted, liquid polymeric solution 135 may be introduced into the cavity of the container 140, such as by pouring, spraying, injection, or the like.

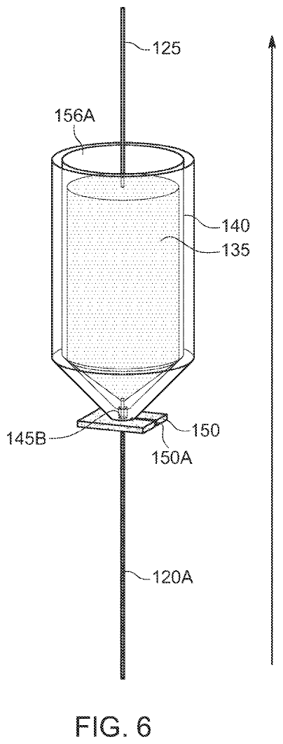

FIG. 6 is a perspective view illustrating removal of the medical device from the container to coat the medical device with a polymeric coating according to one example. With the polymeric solution 135 in the container 140, the portion of the medical device 110 to which the polymeric coating 130 is to be applied may be advanced through the solution 135, such as by pulling upwardly as shown in FIG. 6.

The step of advancing the medical device 110 (or a portion thereof) through the solution 135 may be manually performed or automated. The speed at which the medical device 110 (or a portion thereof) is advanced through the solution 135 may vary but will generally be slow enough to allow the medical device 110 to be adequately coated with the solution. By way of example, the speed of movement may be about 2 inches per minute.

After the solution 135 has been applied to the medical device 110, the coated medical device 100 may be cured or dried so as to remove the solvent and form a solid, continuous polymeric coating 130 on the medical device 110. The manner by which the solution 135 may be cured or dried may vary in different examples. In an example, the coated medical device 100 may be hung on a rack and then heated. An oven or other heating device may be utilized. Use of a hot-air circulation system may aid in improving the uniformity of air temperature to aid in uniformly curing or drying the polymeric coating 130 on the medical device.

In another example, instead of applying the polymeric solution 135 to a medical device 110, the polymeric solution 135 may instead be cured or dried to form all or part of a medical device 110, or to be separately applied to a medical device 110 in a solid state. In an example, the polymeric solution 135 may be extruded into a mold to form either a medical device 110 or a solid coating that may be separately attached to or otherwise applied to a medical device 110. For example, the polymeric solution 135 may be extruded into a tubular form and then allowed to dry or cure, after which the tubular form may be applied to a medical device 110.

The polymeric coating 130 may be applied to a full length of the medical device 110 or to only one or more portions of the length of the medical device 110. In an example, a first portion of the medical device 110 may be coated with the polymeric coating 130 and a second portion of the medical device 110 may not be coated with the polymeric coating 130. In some examples, multiple distinct segments of the medical device 110 may be coated with the polymeric coating 130 with remaining (e.g., intervening) segments not being coated with the polymeric coating 130.

FIGS. 2-6 illustrate an example of a method for applying a solution to a medical device. In the example shown in FIGS. 2-6, it can be seen that the medical device may comprise a guidewire including a core wire and one or more coils which are wound around at least a portion of the length of the core wire. However, the described method may be applied to a wide range of other types of medical devices.

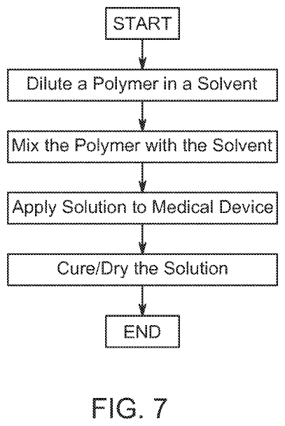

FIG. 7 is a flowchart illustrating a method of coating a medical device with a polymeric coating according to one example. As shown in FIG. 7, the method may comprise the steps of diluting or dissolving the polymer in a solvent, mixing the polymer with the solvent to form a polymeric solution 135, applying the polymeric solution 135 to a medical device 110, and curing or drying the solution to form a solid polymeric coating 130 on the medical device 110 and thereby form the coated medical device 100. The medical device 110 may be cleaned, such as with acetone, and allowed to dry before application of the polymeric solution 135.

In some examples, a hydrophilic coating or solution may be applied to the polymeric coating 130 so as to increase lubricity of the medical device 110. The use of such a hydrophilic coating may be particularly useful with respect to medical devices 110 comprised of guidewires, catheters, and/or pushers. The manner by which the hydrophilic coating may be applied may vary in different examples, including but not limited to dip coating, application with a brush, or the like. Various types of hydrophilic coatings may be utilized, such as but not limited to high lauric acid based hydrophilic materials or compositions. The hydrophilic coating may be applied on top of or around the polymeric coating 130 or may be incorporated therewith.

### Clauses

Clause 1. A polymeric coating for a medical device, comprising: a solvent; and a polyurethane-based polymer dissolved in the solvent.

Clause 2. The polymeric coating of clause 1, wherein the polyurethane-based polymer is comprised of an aliphatic hydrocarbon thermoplastic polyurethane-based solution.

Clause 3. The polymeric coating of clause 1, wherein the solvent is comprised of dimethylacetamide.

Clause 4. The polymeric coating of clause 1, wherein a volume of the polyurethane-based polymer is between about 15%-25% of a total volume of the polymeric coating.

Clause 5. The polymeric coating of clause 1, wherein a volume of the solvent is between about 75%-85% of a total volume of the polymeric coating.

Clause 6. The polymeric coating of clause 1, wherein a volume of the polyurethane-based polymer is about 20% of a total volume of the polymeric coating.

Clause 7. The polymeric coating of clause 6, wherein a volume of the solvent is about 80% of the total volume of the polymeric coating.

Clause 8. The polymeric coating of clause 1, wherein the solvent is comprised of tetrahydrofuran.

Clause 9. The polymeric coating of clause 1, wherein the solvent is comprised of methoxy propyl acetate.

Clause 10. The polymeric coating of clause 1, wherein the solvent is comprised of toluene.

Clause 11. A polymeric coating for a medical device, comprising: a solvent comprised of dimethylacetamide; and a polymer comprised of an aliphatic hydrocarbon thermoplastic polyurethane; wherein a volume of the polymer is between 15%-25% of a total volume of the polymeric coating; and, wherein a volume of the solvent is between 75%-85% of the total volume of the polymeric coating.

Clause 12. A coated medical device system, comprising: a medical device; and a polyurethane-based polymeric solution applied to at least a portion of the medical device.

Clause 13. The coated medical device system of clause 12, wherein the medical device is comprised of a guidewire.

Clause 14. The coated medical device system of clause 13, further comprising a first coil coiled around at least a portion of a length of the guidewire.

Clause 15. The coated medical device system of clause 14, further comprising a second coil coiled around at least a portion of the length of the guidewire.

Clause 16. The coated medical device system of clause 15, wherein the first coil comprises an inner coil and wherein the second coil comprises an outer coil.

Clause 17. The coated medical device system of clause 15, wherein the guidewire is composed of Nitinol.

Clause 18. The coated medical device system of clause 17, wherein the first coil is composed of stainless steel.

Clause 19. The coated medical device system of clause 18, wherein the second coil is composed of platinum tungsten.

Clause 20. The coated medical device system of clause 12, wherein the medical device is comprised of a stent.

Clause 21. A method of fabricating a polymer coated medical device, comprising: diluting a polyurethane-based polymer in a solvent to form a solution; mixing the solution; applying the solution to at least a portion of a medical device; and drying the solution.

Clause 22. The method of clause 21, wherein the step of applying the solution is comprised of: providing a container including an upper opening and a lower opening, the upper opening being wider than the lower opening; inserting the medical device at least partially through the container such that the medical device extends through both the upper opening and the lower opening; at least partially filling the container with the solution; and removing the medical device from the container through the upper opening such that the solution coats at least a portion of the medical device.

Clause 23. The method of clause 22, further comprising the step of attaching a slitted polymer foam or sponge to the lower opening of the container for leak prevention.

Clause 24. The method of clause 21, wherein the step of drying the solution is comprised of hanging the medical device in a rack.

Clause 25. The method of clause 21, wherein the step of drying the solution is comprised of applying heat to the medical device after the solution has been applied.

Clause 26. The method of clause 21, wherein the polyurethane-based polymer is comprised of an aliphatic hydrocarbon thermoplastic polyurethane-based solution.

Clause 27. The method of clause 21, wherein the solvent is comprised of dimethylacetamide.

Clause 28. The method of clause 21, wherein a volume of the polyurethane-based polymer is between 15%-25% of a total volume of the solution.

Clause 29. The method of clause 21, wherein a volume of the solvent is between 75%-85% of a total volume of the solution.

Clause 30. The method of clause 21, wherein a volume of the polyurethane-based polymer is 20% of a total volume of the solution.

Clause 31. The method of clause 30, wherein a volume of the solvent is 80% of the total volume of the solution.

Clause 32. The method of clause 21, wherein the solvent is comprised of tetrahydrofuran.

Clause 33. The method of clause 21, wherein the solvent is comprised of methoxy propyl acetate.

Clause 34. The method of clause 21, wherein the solvent is comprised of toluene.

Clause 35. The method of clause 21, wherein the medical device is comprised of a guidewire.

Clause 36. The method of clause 35, further comprising a first coil coiled around at least a portion of a length of the guidewire.

Clause 37. The method of clause 36, further comprising a second coil coiled around at least a portion of the length of the guidewire.

Clause 38. The method of clause 37, wherein the first coil comprises an inner coil and wherein the second coil comprises an outer coil.

Clause 39. The method of clause 37, wherein the guidewire is composed of Nitinol.

Clause 40. The method of clause 39, wherein the first coil is composed of stainless steel.

Clause 41. The method of clause 40, wherein the second coil is composed of platinum tungsten.

Clause 42. The method of clause 21, wherein the medical device is comprised of a stent.

Clause 43. A polymeric coating for a medical device, comprising: a solvent; and a polycarbonate-based polymer dissolved in the solvent.

Clause 44. A medical device, comprising: a core wire; a first coil wound around the core wire; and a second coil wound around the core wire.

Clause 45. The medical device of clause 44, wherein at least a portion of the first coil is positioned within an inner diameter of at least a portion of the second coil.

Clause 46. The medical device of clause 44, wherein the first coil comprises an inner coil and wherein the second coil comprises an outer coil.

Clause 47. The medical device of clause 44, wherein the first coil comprises a variable pitch along its length.

Clause 48. The medical device of clause 47, wherein the first coil comprises a first segment having a first pitch, a second segment having a second pitch, and a third segment having a third pitch.

Clause 49. The medical device of clause 48, wherein the first pitch is 0.0005 inch, the second pitch is 0.008 inch, and the third pitch is 0.013 inch.

Clause 50. The medical device of clause 49, wherein the first coil comprises a fourth segment having a fourth pitch and wherein the fourth pitch is 0.00175 inch.

Clause 51. The medical device of clause 50, wherein the fourth segment is proximal with respect to the first segment, the second segment, and the third segment.

Clause 52. The medical device of clause 47, wherein the second coil comprises a uniform pitch along its length.

Clause 53. The medical device of clause 52, wherein the uniform pitch of the second coil is 0.00175 inch.

Clause 54. The medical device of clause 52, further comprising a polymeric coating encapsulating the first coil and the second coil.

Although the invention has been described in terms of particular examples and applications, one of ordinary skill in the art, in light of this teaching, can generate additional examples and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1.-54. (canceled)

55. A polymeric coating for a medical device, comprising:
a solvent; and
a polyurethane-based polymer dissolved in the solvent.

56. The polymeric coating of claim 55, wherein the polyurethane-based polymer is comprised of an aliphatic hydrocarbon thermoplastic polyurethane-based solution.

57. The polymeric coating of claim 55, wherein the solvent is comprised of dimethylacetamide.

58. The polymeric coating of claim 55, wherein a volume of the polyurethane-based polymer is between about 15%-25% of a total volume of the polymeric coating.

59. The polymeric coating of claim 55, wherein a volume of the solvent is between about 75%-85% of a total volume of the polymeric coating.

60. The polymeric coating of claim 55, wherein a volume of the polyurethane-based polymer is about 20% of a total volume of the polymeric coating.

61. The polymeric coating of claim 60, wherein a volume of the solvent is about 80% of the total volume of the polymeric coating.

62. The polymeric coating of claim 55, wherein the solvent is comprised of tetrahydrofuran.

63. The polymeric coating of claim 55, wherein the solvent is comprised of methoxy propyl acetate.

64. The polymeric coating of claim 55, wherein the solvent is comprised of toluene.

65. The polymeric coating of claim 55, wherein the solvent is comprised of dimethylacetamide, wherein the polyurethane-based polymer is comprised of an aliphatic hydrocarbon thermoplastic polyurethane, wherein a volume of the polyurethane-based polymer is between 15%-25% of a total volume of the polymeric coating, and wherein a volume of the solvent is between 75%-85% of the total volume of the polymeric coating.

66. The polymeric coating of claim 55, wherein the polyurethane-based polymer is applied to a medical device.

67. A polymeric coating for a medical device, comprising:
a solvent; and
a polycarbonate-based polymer dissolved in the solvent.

68. The polymeric coating of claim 67, wherein the polycarbonate-based polymer is applied to a medical device.

69. A method of fabricating a polymer coated medical device, comprising:
diluting a polyurethane-based polymer in a solvent to form a polymeric coating;
mixing the polymeric coating;
applying the polymeric coating to at least a portion of a medical device; and
drying the polymeric coating.

70. The method of claim 69, wherein the step of applying the polymeric coating is comprised of:
providing a container including an upper opening and a lower opening, the upper opening being wider than the lower opening;
inserting the medical device at least partially through the container such that the medical device extends through both the upper opening and the lower opening;
at least partially filling the container with the polymeric coating; and
removing the medical device from the container through the upper opening such that the polymeric coating coats at least a portion of the medical device.

71. The method of claim 70, further comprising the step of attaching a slitted polymer foam or sponge to the lower opening of the container for leak prevention.

72. The method of claim 69, wherein the solvent is comprised of dimethylacetamide, wherein the polyurethane-based polymer is comprised of an aliphatic hydrocarbon thermoplastic polyurethane, wherein a volume of the polyurethane-based polymer is between 15%-25% of a total volume of the polymeric coating, and wherein a volume of the solvent is between 75%-85% of the total volume of the polymeric coating.

73. The method of claim 69, wherein the medical device is comprised of a guidewire including an inner coil coiled around at least a portion of a length of the guidewire, an outer coil coiled around at least a portion of a length of the inner coil.

74. The method of claim 73, wherein the guidewire is composed of Nitinol, wherein the inner coil is composed of stainless steel, and wherein the outer coil is composed of platinum tungsten.
